(19) United States
(12) Patent Application Publication
(10) Pub. No.: US 2014/0010849 A1
(43) Pub. Date: Jan. 9, 2014

(54) PEANUT ALLERGY TREATMENT

(71) Applicant: Northwestern University, Evanston, IL (US)

(72) Inventors: Daocheng Zhu, Wilmette, IL (US); Robert P. Schleimer, Chicago, IL (US)

(21) Appl. No.: 13/933,312

(22) Filed: Jul. 2, 2013

Related U.S. Application Data
(60) Provisional application No. 61/667,193, filed on Jul. 2, 2012.

(51) Int. Cl. C07K 16/28

(52) U.S. Cl. CPC C07K 16/283
USPC 424/400; 424/134.1

(57) ABSTRACT

The present invention provides compositions, systems, and methods for preventing and treating allergic reactions to peanut exposure. In certain embodiments, the present invention employs fusion proteins comprising a peanut allergen (or allergenic portion thereof), such as Ara h2, and an Fc protein (e.g., Fcγ1 protein), or functional portion thereof.

The present application claims priority to U.S. Provisional application Ser. No. 61/667,193 filed Jul. 2, 2012, which is herein incorporated by reference in its entirety.

This invention was made with government support under grant number R21 AI088808 awarded by the National Institutes of Health. The government has certain rights in the invention.

### FIELD OF THE INVENTION

The present invention provides compositions, systems, and methods for preventing and treating allergic reactions to peanut exposure. In certain embodiments, the present invention employs fusion proteins comprising a peanut allergen (or allergenic portion thereof), such as Ara h2, and an Fc protein (e.g., Fcγ1 protein), or functional portion thereof.

### BACKGROUND

Food allergy is a serious health problem of national importance that is increasing in prevalence worldwide. Peanut is one of the most allergenic foods ¹. Approximately 1.5 million people in the United States have peanut allergy with about 50-100 of those dying each year from the accidental ingestion of foods containing peanuts or peanut extracts ². Affected individuals may experience severe symptoms ranging from urticaria to anaphylaxis. In addition, peanut sensitivity usually appears at an early age and often persists throughout life. Due of the severity of the allergic reaction and the wider use of peanuts as protein extenders in processed foods, the risk to the peanut-sensitive individual is increasing ³. Unlike allergic diseases to inhaled allergens, no effective treatment exists for peanut allergy other than strict avoidance and/or the rapid intervention with epinephrine when reactions do occur ⁴. However, the peanut-hypersensitive population is still at a constant risk of accidentally ingesting peanuts—caused by misleading or absent product labeling, contamination from hidden allergens, and the extensive use of peanut products in processed foods ⁵. Even kissing has been shown to transfer enough peanut antigens to induce a reaction in sensitive subjects ⁶. The ever-present risk of a serious or even fatal reaction takes an enormous emotional toll on the families of children with peanut allergy. Standard subcutaneous immunotherapy that is effective for inhalant allergy has consistently been proven too dangerous in the setting of severe food allergies. Currently, no approved immunotherapy is available for treatment of peanut allergy ⁷.

Peanut allergy is an IgE-mediated hypersensitivity reaction. Peanut-induced allergic reactions are mediated by antigen-specific IgE bound to the high-affinity receptor for IgE (FcγRI) on mast cells, basophils and perhaps other cells. Allergen-induced FcγRI crosslinking causes not only the early phase response, including anaphylaxis, but also the late phase response resulting from the recruitment of inflammatory cells and further release of inflammatory mediators such as cytokines Several novel immunotherapeutic approaches for peanut allergy have been investigated, including peptide immunotherapy, herbal medicine, and mutated protein immunotherapy and anti-IgE therapy. However, the clinical uses of these approaches are limited, either because of safety concerns or lack of efficacy. For example, anti-IgE (Omalizumab) has been tested for its ability to prevent peanut reactions. While showing some level of protection, overall the results were not dramatic in the initial trial and the subsequent trial of anti-IgE in peanut allergy was terminated because of reactions. New therapeutic approaches are urgently needed.

### SUMMARY OF THE INVENTION

The present invention provides compositions, systems, and methods for preventing and treating allergic reactions to peanut exposure. In certain embodiments, the present invention employs fusion proteins comprising a peanut allergen (or allergenic portion thereof), such as Ara h2, and an Fc protein (e.g., Fcγ1 protein), or functional portion thereof. In certain embodiments, the fusion protein comprises at least two peanut allergens (or allergenic portions thereof), such as two Ara h2 proteins, and two Fc proteins (e.g., Fcγ1 proteins), or functional portions thereof (see, e.g., FIG. 1 that has two Ara h2 proteins and two Fc regions).

In some embodiments, the present invention provides methods for preventing or treating an allergic reaction to peanuts comprising: administering a composition to a subject who has been exposed to a peanut allergen such that a peanut induced allergic reaction is reduced or eliminated in the subject, wherein the subject is or is suspected of being allergic to peanuts, and wherein the composition comprises a first fusion protein comprising: i) a first peanut allergen, or first allergenic portion thereof, and ii) an Fc protein (e.g., Fcγ1 protein), or functional portion thereof

In certain embodiments, the present invention provides compositions comprising: a first fusion protein comprising: i) a first peanut allergen, or first allergenic portion thereof, and ii) an Fc protein (e.g., Fcγ1 protein), or functional portion thereof. In other embodiments, the present invention provides systems comprising: i) a composition comprising: a first fusion protein comprising: i) a first peanut allergen, or first allergenic portion thereof, and ii) an Fc protein (e.g., Fcγ1 protein), or functional portion thereof; and ii) an injection device. In particular embodiments, the injection device comprises a self-administered injector.

In certain embodiments, the Fc protein (e.g., Fcγ1 protein, or portion thereof) is human or derived from a human protein (e.g., germline, or taken from the subject themselves). In certain embodiments, the portion of the Fc protein comprises, consists of, or consists essentially of: i) an IgG hinge region (or portion thereof), ii) and IgG CH2 region (or portion thereof), iii) an IgG CH3 region (or portion thereof); or iv) any combination thereof. In certain embodiments, the Fc protein is engineered to contain changes, mutations, or substitutions to enhance the function of the Fc protein. Such Fc engineering is known in the art.

In further embodiments, the first peanut allergen comprises Ara h2 or an allergenic portion thereof. In other embodiments, the first peanut allergen comprises Ara h1 or an allergenic portion thereof. In additional embodiments, the first peanut allergen comprises Ara h3 or an allergenic portion thereof. In certain embodiments, the first peanut allergen is selected from the group consisting of: Ara h1, Ara h2, Ara h3, Ara h4, Ara h5, Ara h6, Ara h7, Ara h8, Ara h9, Ara h10, Ara h11, or an allergic reaction inducing portion of Ara h1, Ara h2, Ara h3, Ara h4, Ara h5, Ara h6, Ara h7, Ara h8, Ara h9, Ara h10, Ara h 11. It is noted that allergenic portions of these proteins can be found, for example, by replacing the full length Ara h2 protein in the Examples below with the candidate portion to determine if the candidate portion functions to block or reduce the allergic reaction (e.g., in a manner similar to the full length Ara h2 in the Examples).

In some embodiments, the peanut induced allergic reaction comprises peanut-induced anaphylaxis. In further embodiments, administering the compositions reduces or eliminates the peanut-induced anaphylaxis in the subject. In particular embodiments, the peanut induced allergic reaction comprises airway inflammation. In some embodiments, the administering reduces or eliminates the airway inflammation in the subject.

In particular embodiments, the compositions further comprise a physiologically tolerable buffer and/or agent used to treat peanut allergies (e.g., an anti-histamine). In further embodiments, the compositions further comprise a second fusion protein, which comprises: i) a second peanut allergen, or second allergenic portion thereof, different from the first peanut allergen, and ii) a Fcγ1 protein, or functional portion thereof. In particular embodiments, the first peanut allergen comprises Ara h2 or an allergenic portion thereof, and wherein the second peanut allergen comprises Ara h1 or Ara h3, or an allergenic portion thereof. In other embodiments, the compositions are located in a self-administered injector.

### DESCRIPTION OF THE FIGURES

FIGS. 1A-B. Diagram of AHG2-induced inhibition. (A) Hypothetical computerized 3D structure of dimerized AHG2. (B) Proposed mechanism by which AHG2 inhibits FcεRI-meditated degranulation. It is noted that the present invention is not limited to any particular mechanism and an understanding of the mechanism is not necessary to practice the invention.

FIGS. 2A-C. Characterization of AHG2 fusion protein. (A) AHG2 was expressed with the correct size. AHG2 was run on SDS-PAGE gel and then probed with antibodies specific for human IgG Fc and Ara h2. (B) AHG2 was recognized by peanut-specific IgE. ELISA plate was coated with AHG2 or WPE as indicated, loaded with peanut allergy patient serum and probed with anti-human IgE conjugated with Alkaline Phosphatase. (C) AHG2 bound to FcγRIIb expressed on HMC-1 cells. The cells were incubated with IgG or AHG2 and stained with anti-human IgG labeled with FITC. The cells were then run through flow cytometry (BD LSRII, BD FACSDiva).

FIG. 3. AHG2 blocked Ara h2-induced vascular leak in transgenic mice. (A) WPE induced peanut specific IgE-mediated PCA in transgenic mice. The mouse was sensitized with peanut allergy patient serum (I); non allergic serum (II); NP-specific IgE (III); and heat-activated peanut allergy patient serum (IV). After four hours, the mouse was challenged with WPE. (B) Ara h2 induced peanut specific IgE-meditated PCA in transgenic mice. The mouse was sensitized with peanut allergy patient serum (I), 1:1 diluted serum (II), 1:5 diluted serum (III), and 1:10 diluted serum (IV). After four hours, the mouse was challenged with 50 μg of purified peanut allergen Ara h2. (C) AHG2 did not induce peanut specific IgE-mediated PCA in transgenic mice. The mouse was sensitized with peanut allergy patient serum (I), 1:1 diluted serum (II), 1:5 diluted serum (III) and 1:10 diluted serum (IV). After four hours, the mouse was challenged with 100n of AHG2. (D) AHG2 inhibited Ara h2-induced peanut specific IgE-mediated PCA in transgenic mice. The mouse was sensitized with peanut serum (I); or serum plus 10 μg of AHG2 (II); or serum plus 1 μg of AHG2 (III) and serum plus 0.1 μg of AHG2 (IV). After four hours, the mouse was challenged with 100 μg of purified Ara h2. Each experiment was repeated with three transgenic mice.

FIGS. 4A-C. AHG2 inhibited WPE-induced allergic reactions in vitro and in vivo. (A) AHG2 inhibited histamine release in human basophils. Purified human basophils were treated with different doses of AHG2 and then challenged with WPE. Histamine from the supernatant was quantified. Results are representative of 3 separate experiments, each done in duplicate. (B) AHG2 inhibited PCA reaction in transgenic mice. A transgenic mouse expressing the human FcεRIα was sensitized with I: peanut allergic patient serum; II: patient serum with 10 ng of AHG2; III: patient serum with 100 ng of AHG2; IV: patient serum with 1 μg of AHG2 and challenged with WPE four hours later. (C) The average blue density of five mice for each of the above tests.

FIGS. 5A-C. AHG2 inhibited systemic anaphylaxis caused by the peanut extract in mice. Four groups of mice were sensitized with the peanut antigen. Group 1(n=5) was a control; Group 2(n=9) was challenged with WPE; Group 3 (n=9) was treated with 1 mg/kg of AHG2 (AHG2-L) and then challenged with WPE; Group 4 (n=9) was treated with 10 mg/kg of AHG2 (AHG2-H) and then challenged with WPE. Each mouse was evaluated for their symptoms score (A), body temperature (B), and histamine level in blood (C). Data are representative of two separate experiments. *, P<0.05

FIG. 6. AHG2 lost its inhibitory effect in FcγRIIb deficient mice. Three groups of FcγRIIb KO mice were sensitized and challenged with WPE. Group 1 (n=6) was control; Group 2(n=6) was not treated with AHG2; Group 3 (n=7) was treated with 10mg/kg of AHG2 before WPE challenge. Each mouse was evaluated for their symptoms score (A) and body temperature (B). Data are representative of two separate experiments.

FIGS. 7A-B. AHG2 inhibited WPE-induced inflammation in the airways of WPE-sensitized mice. A, Differential cell counts in bronchoalveolar lavage fluid. The bronchoalveolar lavage fluid was collected from each mouse of the above groups. The results are representative of the average of each group. *P<0.05. B, Histologic airway changes. Representative histologic sections of lung tissues from the mice in FIG. 5 were stained with hematoxylin and eosin. Bar=100 μm.

FIG. 8. AHG2 fusion protein was recognized by peanut-specific IgE from serum from a patient with peanut allergy (no. 15815). M, Molecular weight.

FIG. 9. Serum levels of specific IgE to WPE and the peanut components in 3 patients with peanut allergy.

FIG. 10. AHG2 inhibited histamine release in RBL-2H3 cells transfected with human FcεRIα and FcγRIIb.

FIG. 11. AHG2 inhibited PCA reaction in transgenic mice sensitized with patients' sera no. 17019 (A) and no. 18885 (B). PS, Serum from a patient with peanut allergy; PS-0.01, serum with 0.01 μg of AHG2; PS-0.1, serum with 0.1 μg of AHG2; PS-1, serum with 1 μg of AHG2.

FIG. 12. AHG2 inhibited systemic anaphylaxis caused by the peanut extract in the sensitized mice. After challenge with WPE, the sensitized mice were randomly divided into 2 groups: one group (n=8) was treated with our AHG2 protein, and the other (n=8) was treated with PBS as a control. After treatment, all mice were intraperitoneally challenged again with WPE. Each mouse was evaluated in terms of symptoms (A), and body temperature was measured (B).

### DEFINITIONS

As used herein, the term “Fc region” refers to a C-terminal region of an immunoglobulin heavy chain. The “Fc region” may be a native sequence Fc region or a variant Fc region. Although the generally accepted boundaries of the Fc region of an immunoglobulin heavy chain might vary, the human IgG heavy chain Fc region is usually defined to stretch from an amino acid residue at about position Cys226, or from Pro230, to the carboxyl-terminus thereof. In some embodiments, variants comprise only portions of the Fc region and can include or not include the carboxyl-terminus. The Fc region of an immunoglobulin generally comprises two constant domains, CH2 and CH3. In some embodiments, variants having one or more of the constant domains are contemplated. In other embodiments, variants without such constant domains (or with only portions of such constant domains) are contemplated in the fusion proteins of the present invention.

As used herein, the “CH2 domain” (also referred to as “Cγ2” domain) generally comprises the stretch of residues that extends from about amino acid 231 to about amino acid 340 in an Fc region (e.g. in the human IgG Fc region). The CH2 domain is unique in that it is not closely paired with another domain. Rather, two N-linked branched carbohydrate chains are interposed between the two CH2 domains of an intact native IgG molecule.

As used herein, the “CH3 domain” (also referred to as “Cγ3” domain) generally comprises the stretch of residues C-terminal to a CH2 domain in an Fc region (e.g., from about amino acid residue 341 to about amino acid residue 447 of a human IgG Fc region).

As used herein, the term “hinge region” generally refers to the stretch of amino acids in human IgG1 stretching from about Glu216 to Pro230 of human IgG1. Hinge regions of other IgG isotypes may be aligned with the IgG1 sequence by placing the first and last cysteine residues forming inter-heavy chain S—S bonds in the same positions.

As used herein, an Fc region may possess “effector functions” that are responsible for activating or diminishing a biological activity (e.g. in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc.

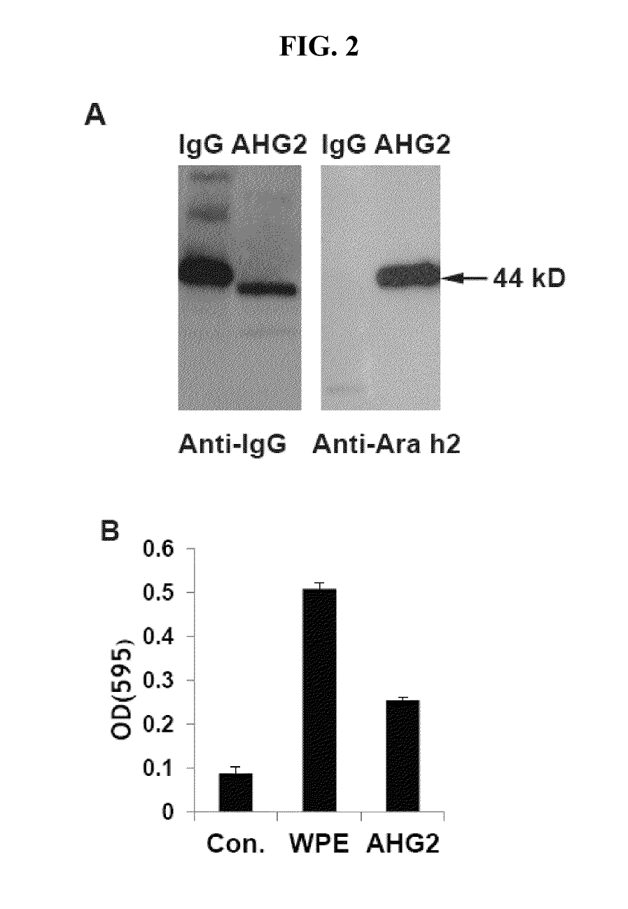

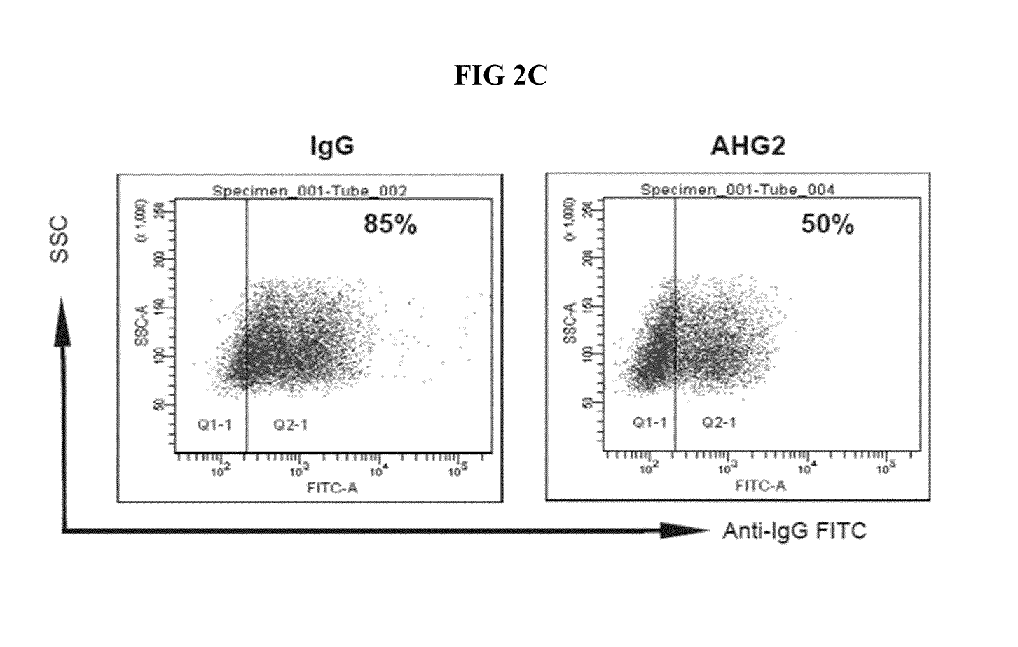

As used herein the term “native sequence Fc region” or “wild type Fc region” refers to an amino acid sequence that is identical to the amino acid sequence of an Fc region commonly found in nature. Exemplary native sequence human Fc regions are shown in FIG. 2 of U.S. Pat. Pub. US20080089892 (herein incorporated by reference) and include a native sequence human IgG1 Fc region (f and a,z allotypes); native sequence human IgG2 Fc region; native sequence human IgG3 Fc region; and native sequence human IgG4 Fc region as well as naturally occurring variants thereof. Other sequences are contemplated and are readily obtained from various web sites (e.g., NCBI's web site).

As used herein, the term “variant Fc region” refers to amino acid sequence that differs from that of a native sequence Fc region (or portions thereof) by virtue of at least one amino acid modification (e.g., substitution, insertion, or deletion), including heterodimeric variants in which the heavy chain subunit sequences may differ from one another. In certain embodiments, the variant Fc region has at least one amino acid substitution compared to a native sequence Fc region (e.g. from about one to about ten amino acid substitutions, and or from about one to about five amino acid substitutions in a native sequence Fc region). In particular embodiments, variant Fc regions will possess at least about 80% homology with a native sequence Fc region, preferably at least about 90% homology, and more preferably at least about 95% homology.

As used herein, an “amino acid modification” refers to a change in the amino acid sequence of a given amino acid sequence. Exemplary modifications include, but are not limited to, an amino acid substitution, insertion and/or deletion. In some embodiments, the amino acid modification is present in the peanut allergen protein (e.g., Ara h1, Ara h2, Ara h3, etc.), in the Fc region peptide (e.g., Fcγ1), or in both.

As used herein, an “amino acid modification at” a specified position (e.g. in the Fc region or a peanut allergen) refers to the substitution or deletion of the specified residue, or the insertion of at least one amino acid residue adjacent the specified residue. By insertion “adjacent” a specified residue is meant insertion within one to two residues thereof The insertion may be N-terminal or C-terminal to the specified residue.

As used herein, an “amino acid substitution” refers to the replacement of at least one existing amino acid residue in a given amino acid sequence with another different “replacement” amino acid residue (e.g., in an Fc region peptide or a peanut allergen peptide, or both). The replacement residue or residues may be “naturally occurring amino acid residues” (i.e. encoded by the genetic code) and selected from: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline

(Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues (s) in a polypeptide chain. Examples of non-naturally occurring amino acid residues include norleucine, ornithine, norvaline, homoserine and other amino acid residue analogues such as those described in Ellman et al. Meth. Enzym. 202: 301-336 (1991), herein incorporated by reference.

As used herein, the term “amino acid insertion” refers to the incorporation of at least one amino acid into a given amino acid sequence (e.g., into a peanut allergen peptide and/or an Fc region peptide). In preferred embodiments, an insertion will usually be the insertion of one or two amino acid residues. In other embodiments, the insertion includes larger peptide insertions (e.g. insertion of about three to about five or even up to about ten amino acid residues. As used herein, the term “amino acid deletion” refers to the removal of at least one amino acid residue from a given amino acid sequence. The peanut allergens and/or Fc regions of the present invention may contain amino acid insertions.

### DETAILED DESCRIPTION

The present invention provides compositions, systems, and methods for preventing and treating allergic reactions to peanut exposure. In certain embodiments, the present invention employs fusion proteins comprising a peanut allergen (or allergenic portion thereof), such as Ara h2, and an Fc protein (e.g., Fcγ1 protein), or functional portion thereof.

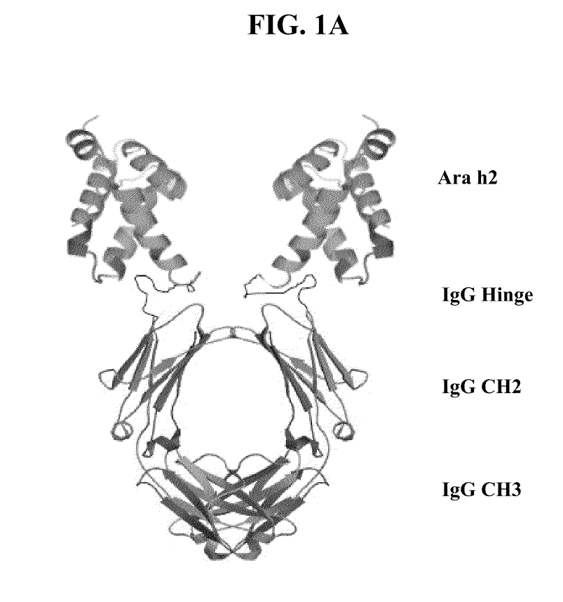

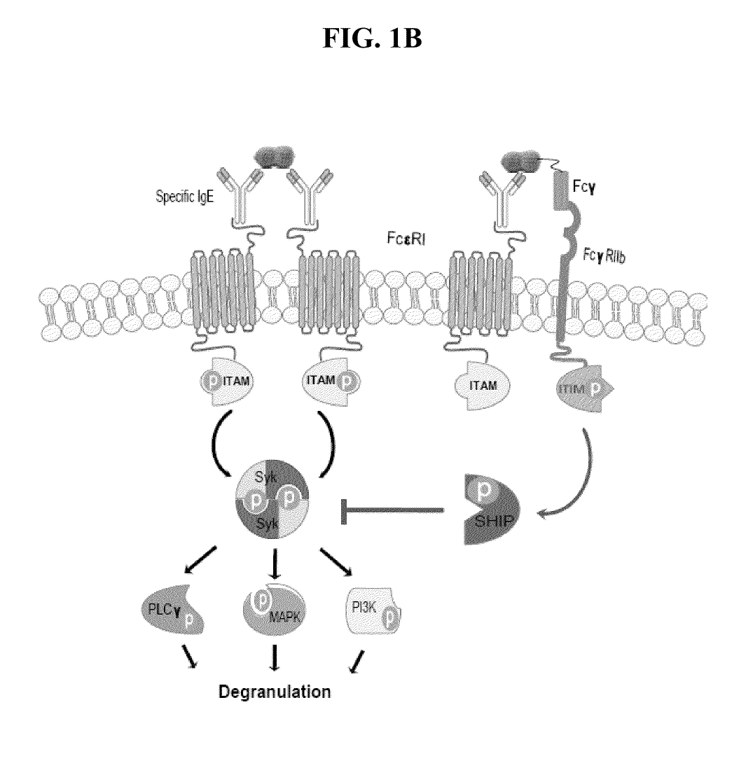

In certain embodiments, the present invention provides a safe immunotherapy that comprises a peanut-human fusion protein composed of the major peanut allergen Ara h2 (Koppelman et al., Clin Exp Allergy 2004;34:583-90, herein incorporated by reference) and human IgG Fcγ1 (FIG. 1A). While the present invention is not limited to any particular mechanism, and an understanding of the mechanism is not necessary to practice the present invention, it is believed that peanut allerg-Fcy fusion proteins will inhibit WPE-induced allergic reactions by indirectly cross-linking inhibitory FcγRIIb with peanut-specific IgE bound to FcεRI (FIG. 1B). This concept is based upon the fact that peanut allergy is an IgE-mediated hypersensitivity reaction ⁹ and during responses, cross-linking of the high affinity IgE receptors (FcεRI) via IgE bound to multivalent peanut antigen results in the activation of degranulation of mast cells and basophils ^{10,11}. Previous studies have demonstrated that aggregating the inhibitory receptor FcγRIIb with FcεRI leads to the inhibition of degranulation ¹²⁻¹⁴. Work conducted during development of embodiments of the present invention found that using an Fcγ-allergen construct to indirectly cross-link FcγRIIb and FcεRI through the antigen-specific IgE also inhibits degranulation.

As described in the Examples below, work was conducted during development of embodiments of the present invention which genetically designed and expressed a novel plant-human fusion protein composed of the major peanut allergen Ara h2 and human IgG Fcγ1. The Ara h2-Fcγ fusion protein (AHG2)'s function was tested in purified human basophils. Transgenic mice expressing human FcεERIα and a peanut allergy murine model were also used. It was found that AHG2 inhibited histamine release induced by whole peanut extract (WPE) from basophils of peanut allergic subjects while the fusion protein itself failed to induce mediator release. AHG2 inhibited the WPE-induced peanut-specific IgE-mediated passive cutaneous anaphylaxis (PCA) in hFcεRIα transgenic mice. AHG2 also significantly inhibited acute anaphylactic reactivity including the prototypical drop of body temperature in WPE-sensitized mice challenged with crude peanut extract. Histologic evaluation of the airways showed that AHG2 decreased peanut-induced inflammation while the fusion protein itself did not induce airway inflammation in peanut sensitized mice. AHG2 did not exert an inhibitory effect in mice lacking the FcγRII. Therefore, the Examples below showed that AHG2 inhibited peanut-specific IgE-mediated allergic reactions in vitro and in vivo.

The compositions and methods of the present invention may be used to treat peanut allergy. Peanut allergy is caused by multiple antigens. Unlike cat allergy, which is caused by the sole allergen Fel d1, eight proteins in peanut have been implicated as allergens. Fortunately, there are three major human allergens in peanut, Ara h1, Ara h2 and Ara h3, to which more than 90% of peanut hypersensitive individuals react. All of these allergens have been reasonably well characterized and cloned. Ara h1 is a 63.5 kDa glycoprotein that comprises 12-16% of total peanut proteins, and has a high frequency of sensitization. DNA sequence analysis of Ara h1 revealed that Ara h1 allergen has significant homology with the vicilin family of seed storage proteins of other legumes, such as soybean, pea and common bean. Ara h2 is a glycoprotein of about 17 kDa with at least 2 major bands on electrophoresis and an isoelectric point of 5.2. The amino acid sequence of Ara h2 protein is composed of a high percentage of glutamic acid, aspartic acid, glycine, and arginine. Sequence analysis of Ara h2 protein showed similarity to seed storage proteins of the conglutin family, and the protein has at least 10 IgE epitopes. Ara h3 is a 57 kDa protein and belongs to the glycinin storage protein family. It can be recognized by 45% patients with peanut hypersensitivity. Recently, it was found that peanut-derived Ara h6 is a 15 kDa biological active allergen recognized by the majority of the peanut-allergic patient population. Because Ara h6 is homologous to Ara h2 to a large extent, IgE-binding to Ara h6 is cross-reactive with Ara h2.

The present invention is not limited by the type of plant allergen present in the allergen-Fcγ fusion protein. In certain embodiments, the allergen protein is selected from the group consisting of: Ara h1, Ara h2, Ara h3, Ara h4, Ara h5, Ara h6, Ara h7, Ara h8, Ara h9, Ara h10, and Ara h11 or an allergenic portion thereof. Accession numbers for the amino acid sequences for these proteins are as follows: Ara h1 (ACF22884), Ara h2 (AAN77576), Ara h3 (ABI17154), Ara h4 (AAD47382), Ara h6 (AF092846 1), Ara h7 (ABW17159), and Ara h8 (AAQ91847), all of which are herein incorporated by reference. In certain embodiments, combinations of any or all of these fusion proteins (with different plant proteins) are used together to treat allergic reactions (e.g., allergic reactions to peanuts).

### EXAMPLES

### Example 1

### Generating AraH2-Fcγ1 Constructs

This example describes generating AraH2-Fcγ1 constructs, which are composed of a major peanut allergen Ara h2 and the human IgG Fcγ1. First, Ara h2 cDNA was fused to genomic DNA of Fcγ1 (hinge-CH2-CH3) and then the chimeric gene was cloned into a mammalian expression vector pSecTag2 (INVITROGEN). After transfection, the fusion protein

Ara h2-Fcγ was expressed in the supernatants of CHO cell cultures. Western Blot showed that this chimeric protein was expressed as the predicted dimer of approximately 88 KD. The purified Ara h2-Fcγ fusion protein reacted with both specific anti-human IgG Fc and anti-Ara h2 antibodies. Further results showed that the fusion protein bound in a fashion similar to native IgG to FcγRIIb expressed on HMC-1 cells and peanut-specific IgE from patient serum recognized the fusion protein through binding to the peanut antigen Ara h2.

### Example 2

### Testing AraH2-Fcγ1 Constructs

This example describes testing of the AraH2-Fcγ1 constructs described in Example 1, including their function in purified human basophils and transgenic peanut allergy murine model.

### Methods

### Mice and Reagents

Six-week-old female C57BL/6 and Fcgr2b^{tmiTtk} mice were purchased from The Jackson Laboratory (Bar Harbor, Me.). They were maintained on peanut-free chow under specific pathogen-free conditions. Standard guidelines for the care and use of animals were followed. WPE (whole peanut extract) was purchased from the Greer (Lenoir, N.C.). The purified peanut allergen Ara h2 and anti-Ara h2 antibody were purchased from Indoor Biotechnologies (Charlottesville, Va.). The cDNA for the major peanut allergen Ara h2 was kindly provided by Dr. Steve Stanley, University of Arkansas School of Medicine, Little Rock, Ark.

### Purification of Human Basophils

Human basophils were purified by Ficoll gradient centrifugation (GE Healthcare, Piscataway, N.J.), followed by negative selection using magnetic beads (Miltenyi Biotec, Auburn, Calif.). Basophil purities were >90% as determined by both Acid Toluidine Blue staining and FACS analysis (BD LSRII, BD FACSDiva) with CD203c and CD123 antibodies ¹⁶. Informed consent was obtained from all human subjects as approved by the Institutional Review Board at Northwestern University.

### Passive Cutaneous Anaphylaxis in Human FcεRIα Chain Transgenic Mice

The human FcεRIα chain transgenic mice were kindly provided by Dr Jean-Pierre Kinet ¹⁷. The mice were intradermally injected with 50 μL of serum from a patient with peanut allergy (51.3 kVA) (No. 15815, Plasmalab) to sensitize the skin mast cells. The different doses of AHG2 were added to the patient serum prior to passive sensitization. Four hours later, the mice were challenged intravenously with 100 μg of WPE plus 1% Evan blue in a volume of 200 μL. The mice were killed 30 minutes after the intravenous challenge.

### Peanut Allergy Mouse Model

Mice were sensitized as described by Jordana ¹⁸, but with slight modifications. The mice were orally sensitized with 500 μg of WPE along with 10 μg of cholera toxin (List Biological Laboratories) once a week for four weeks. Sensitized mice were intravenously challenged with 100 μg of WPE two weeks after the last sensitization. Before the challenge, sensitized mice were subcutaneously treated with different doses of AHG2. The airway tissues were collected two days after the challenge.

### Assessment of Hypersensitivity Reactions

Anaphylactic symptoms were evaluated 30 minutes after the challenge dose utilizing a defined scoring system (Table I). Scoring of symptoms was performed in a blinded manner by 3 independent investigators.

### Measurement of Core Body Temperatures

After the challenge, body temperatures were measured every 10 min with a rectally inserted thermal probe (Physitemp Instruments Inc, Clifton, N.J.).

### Statistics

All data are reported as the mean±SEM unless otherwise stated. Differences between groups were analyzed using the software Graphpad Prism 5.0. A p value of less than 0.05 was considered significant.

### RESULTS

### AHG2 Inhibited WPE-Induced Histamine Release and Degranulation

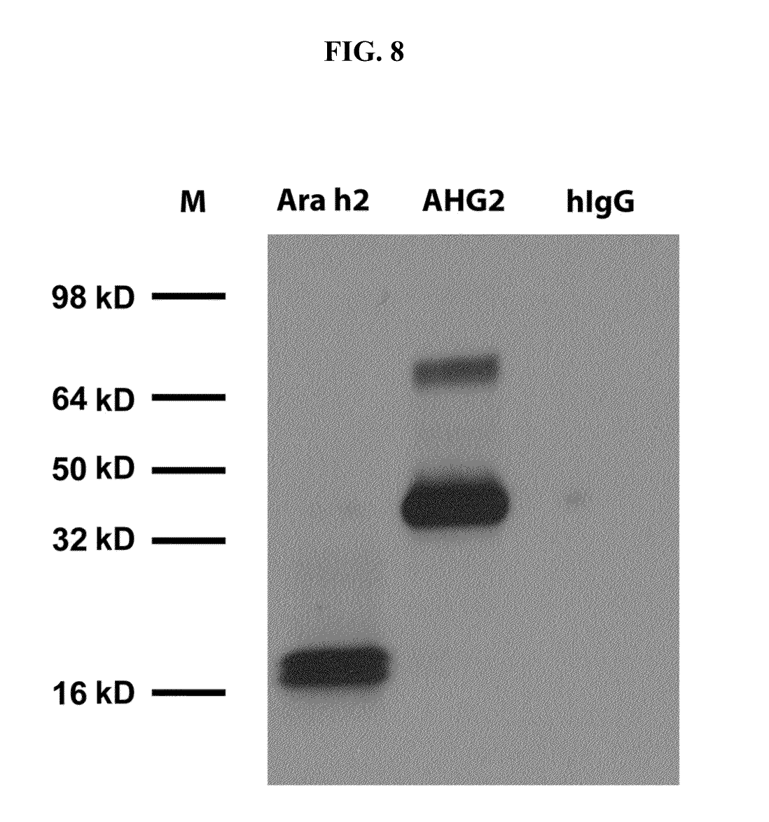

The AHG2 fusion protein was constructed as described in Example 1, being composed of the major peanut allergen Ara h2 plus the hinge, CH2 and CH3 of human gamma 1. Western blotting confirmed that AHG2 had the predicted size of 44 kDa. The amino acid sequence of AHG2 was confirmed by mass spectrometry. Further probing found that AHG2 was recognized by both monoclonal anti-Ara h2 and anti-human IgG Fc specific antibodies (FIG. 2A). In addition, the ELISA result showed that the peanut allergen Ara h2 component of AHG2 was recognized by peanut specific IgE (FIG. 2B, and see FIG. 8). Flow cytometry showed that AHG2 binds with FcγRIIb expressed on HMC-1 cells (FIG. 2C). These results demonstrate that the fusion protein AHG2 was expressed properly, having both peanut allergen and Fcγ antigenic or functional moieties intact.

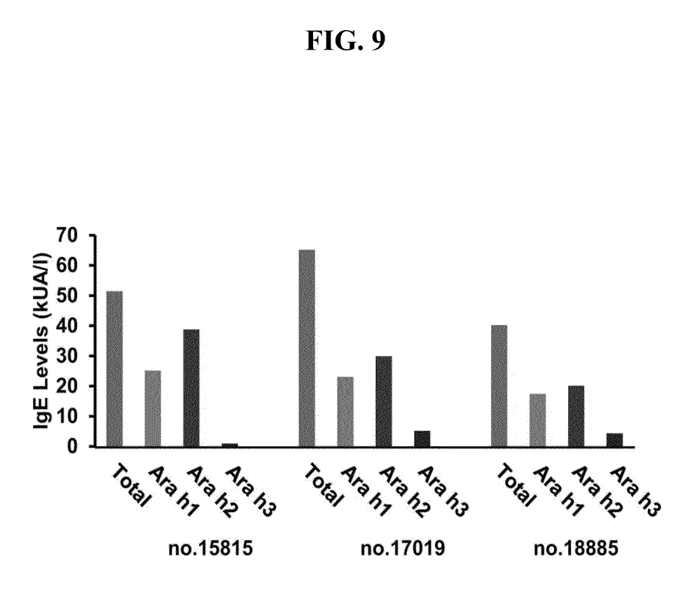

To confirm that AHG2 itself does not cause an allergic reaction, AHG2 was tested on human basophils in vitro and humanized transgenic mice in vivo. Primary human basophils were purified from the white blood cell filters using a Basophil Isolation Kit (Miltenyi Biotec), according to the manufacture's methodology. The purity of basophils was determined to be >90% by both Acid Toluidine Blue staining and FACS analysis with CD203c and CD123 antibodies. The purified basophils were sensatized with peanut allergic patient serum (51.3 kU/l) (No. 15815, Plasmalab), as well as serum from two other patients with peanut allergy (no. 18885 and 17019), see FIG. 9. After 24 hours, the sensitized cells were washed and treated with different doses of AHG2 for one hour. The supernatants were then collected to determine histamine levels. The results show that AHG2 did not induce any detectable histamine release from human basophils, while WPE did.

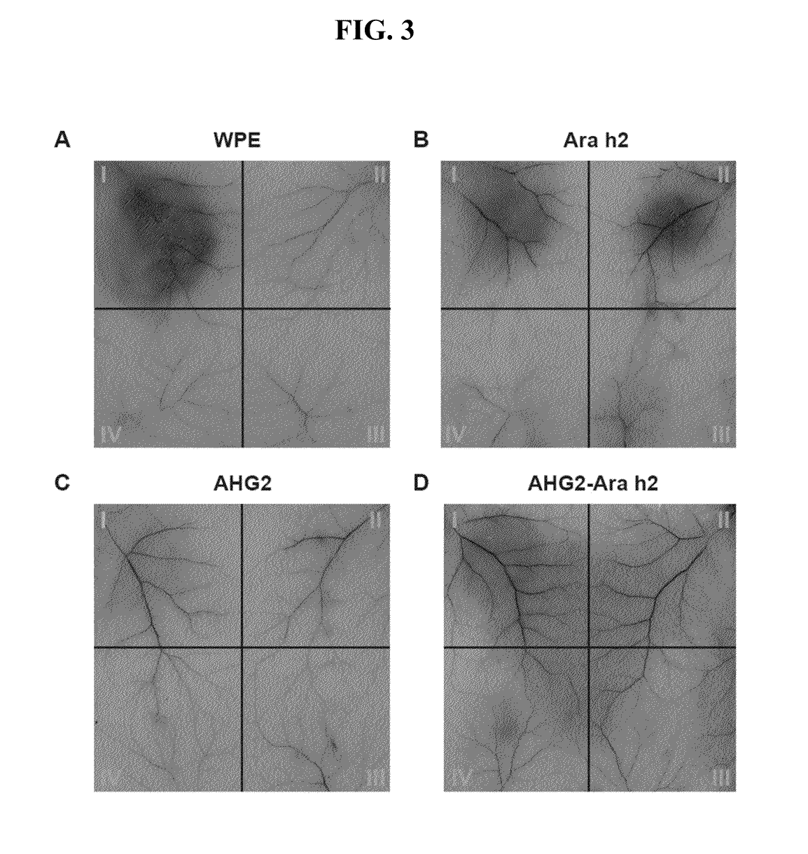

Before testing AHG2 in vivo, a peanut specific IgE-mediated passive cutaneous anaphylaxis (PCA) model was set up in transgenic mice that expressed human IgE receptors ¹⁷. Each mouse was intradermally sensitized with 50 μl of peanut-allergic patient serum, normal human serum, anti-NP IgE, and heat inactivated peanut-allergic patient serum. After four hours, mice were then intravenously challenged with 100 μl of WPE plus 1% Evans blue. Thirty minutes later, the size of the area of bluing at each site was measured to determine the intensity of the passive cutaneous anaphylaxis. It was found that WPE induced degranulation solely in peanut-specific IgE-sensitized mice (FIG. 3A).

To test the fusion protein in vivo, human FcεRIα transgenic mice were intradermally sensitized with 50 μl of various concentrations of peanut-allergic serum. Four hours later, mice were then intravenously injected with 50 μg of purified Ara h2 or 100 μg of AHG2 (that carried an equivalent amount of Ara h2) with 1% Evans blue. It was found that the peanut allergen Ara h2 induced degranulation (shown as vascular leak indicated by blue skin) while AHG2 did not (FIG. 3B and 3C). The transgenic mouse sensitized with peanut allergic patient serum plus different doses of AHG2 was intravenously challenged with peanut allergen Ara h2 four hours later. It was found that 1 μg of AHG2 completely blocked Ara h2-induced vascular leak (FIG. 3D). In addition, it was demonstrated that AHG2 did not block 4-hydroxy-3-nitrophenylacetyl Bovine Serum Albumin (NP-BSA)-induced NP specific IgE-mediated PCA reaction in transgenic mice. These results indicate that AHG2-induced inhibition is peanut-specific.

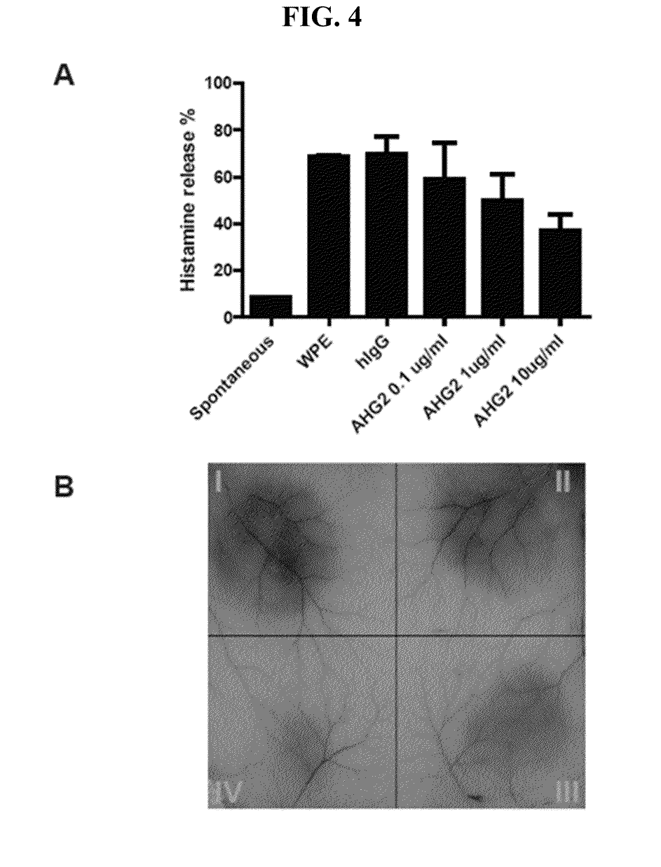

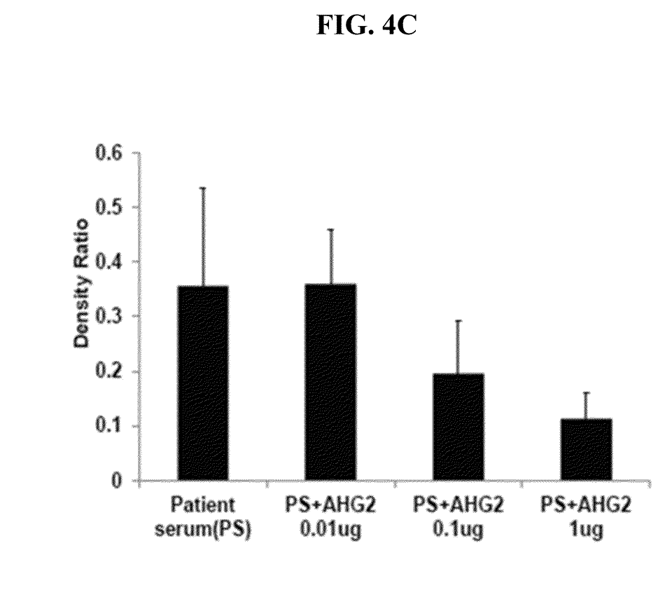

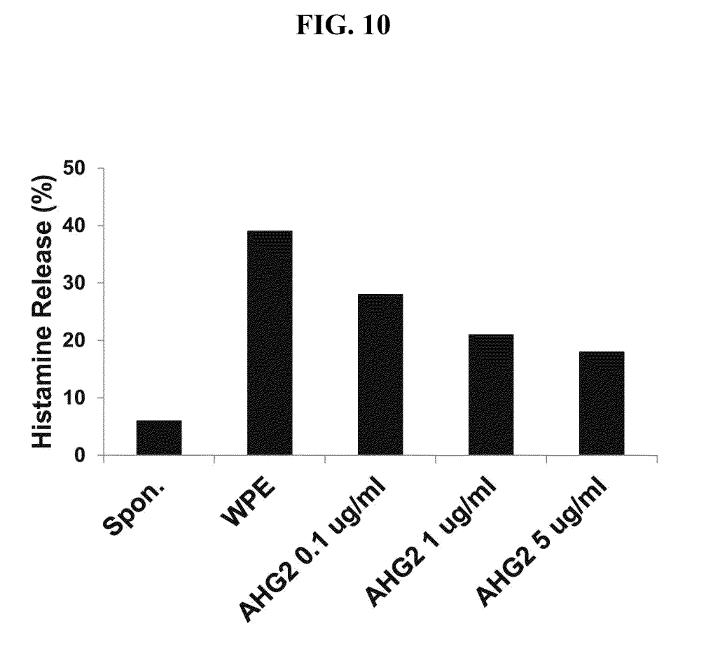

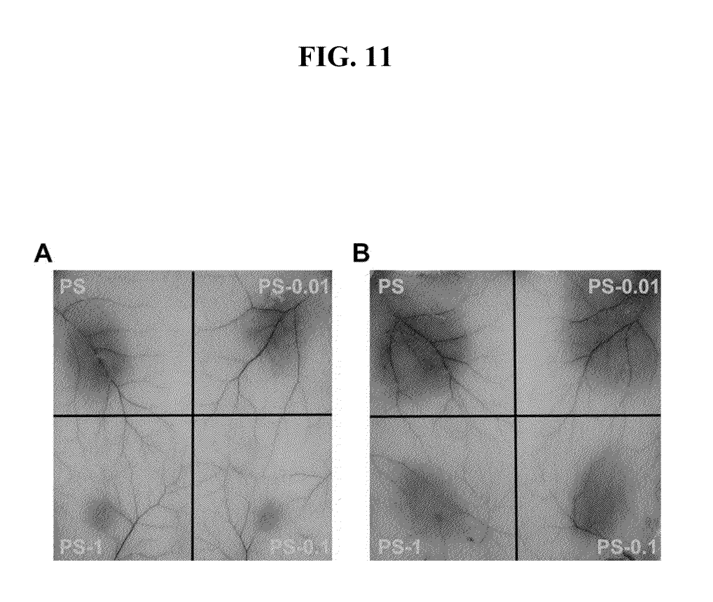

Although these results show that AHG2 completely blocked Ara h2-induced degranulation it should preferably block the antigen-induced allergic reaction to WPE. To test this, purified human basophils were sensitized with peanut allergic patient serum. After 24 hours, the cells were washed and treated with different doses of the AHG2 fusion protein. Two hours later, the cells were challenged with WPE. After thirty minutes, the supernatants were immediately collected and the histamine concentration quantitated using ELISA. The results showed that the AHG2 fusion protein partially inhibited WPE-induced, peanut-specific IgE-mediated histamine release in human basophils in a dose-dependent manner (FIG. 4A). Similar results were found when AHG2 was tested in RBL-2H3 cells sensitized with sera from 2 different patients with peanut allergy (see FIG. 10). Furthermore, it was found that AHG2 significantly inhibited WPE-induced degranulation in human FcεRIα transgenic mice (FIG. 4B). The transgenic mice were intradermally sensitized with 50 μl of the peanut-allergic serum alone and in combination with different doses of AHG2. Four hours later, the mice were given an intravenous challenge with WPE (0.5 mg) plus 1% Evans blue. The result showed that AHG2 dose-dependently inhibited degranulation induced by WPE in peanut specific IgE sensitized transgenic mice (FIG. 4B). Consistent results were obtained from testing AHG2 in groups of five mice (FIG. 4C). Similar results were produced when we sensitized the same mice with sera from 2 different patients with peanut allergy (no. 17019 and no. 18885, see FIG. 11).

AHG2 inhibited peanut-induced acute systemic anaphylaxis in mice

Using a murine peanut allergy model ¹⁸, acute severe systemic anaphylaxis was induced by intravenously challenging the oral peanut sensitized mice. Female C57BL/6 mice were orally sensitized with 500 μg of WPE plus 10 μg of Cholera toxin in 100 μl of saline once a week for four weeks. Two weeks after the last sensitization, the mice were intravenously challenged with 100 μg of WPE. The clinical symptoms were evaluated by three experienced investigators using a scoring system (Table 1).

TABLE I
Anaphylactic symptom score table.

| Score | Symptoms |
|---|---|
| 0 | No clinical symptoms |
| 1 | Repetitive mouth/ear scratching and ear canal digging with hind |
| | legs |
| 2 | Decreased activity; self isolation; puffiness around eyes and/or |
| | mouth |
| 3 | Periods of motionless for more than 1 min; lying prone on stomach |
| 4 | No response to whisker stimuli; reduced or no response to prodding |
| 5 | Endpoint: trem or; convulsion; death |

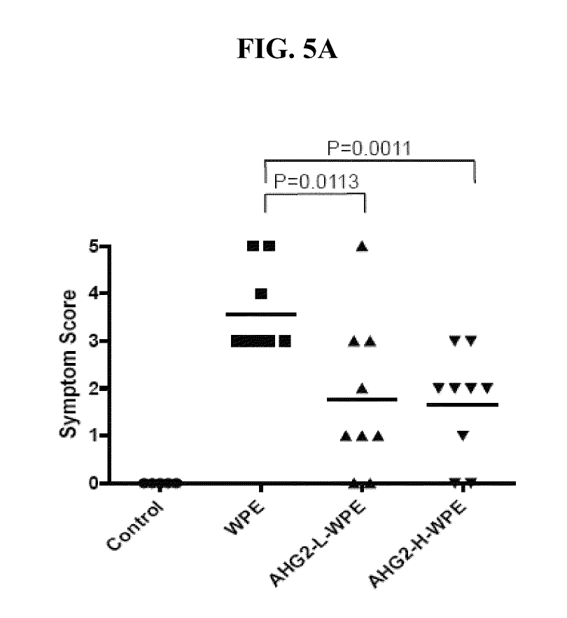

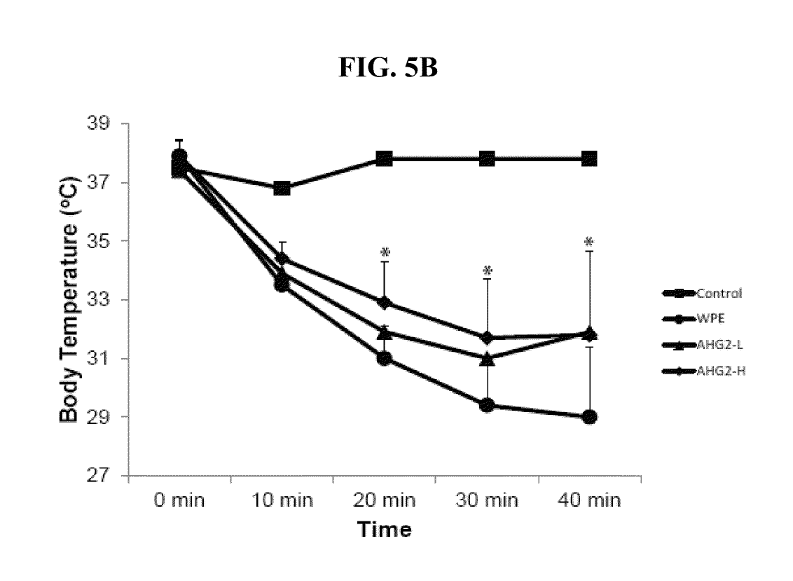

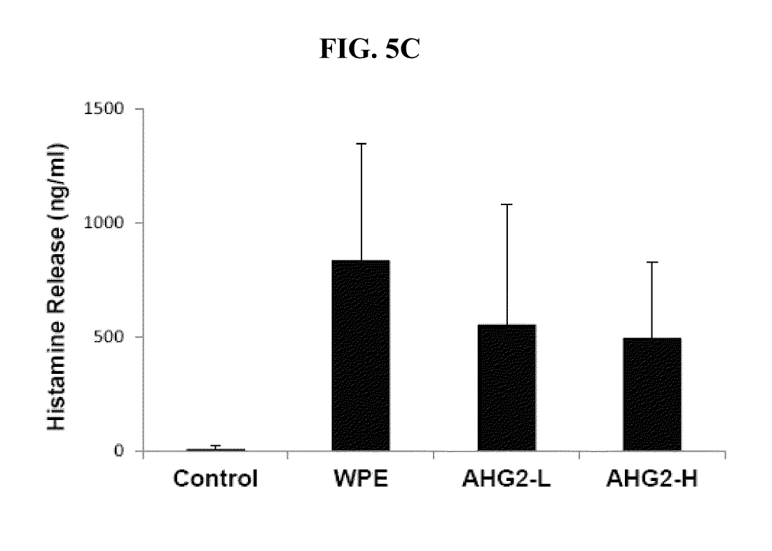

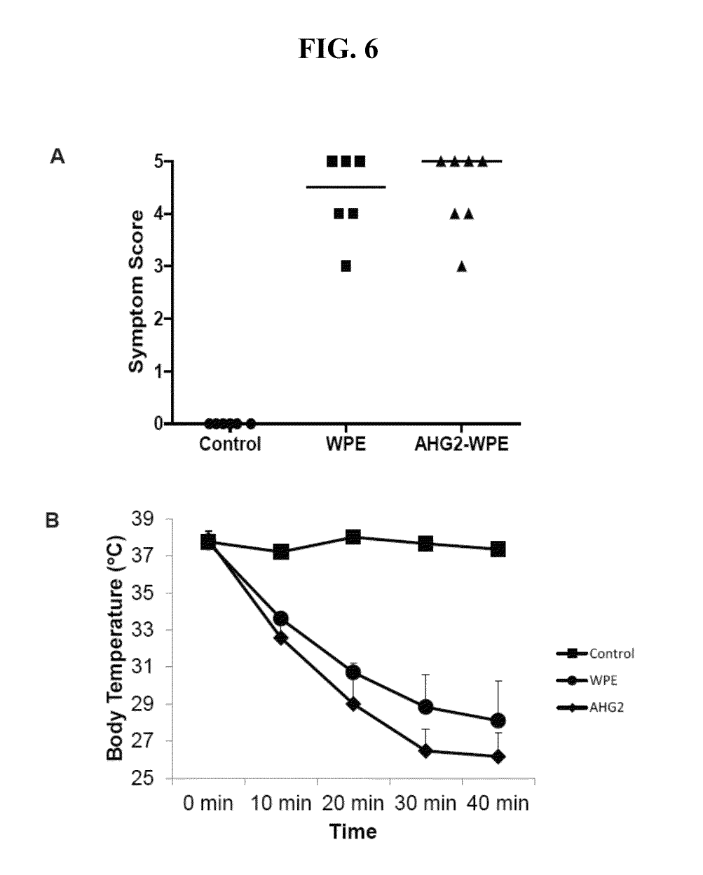

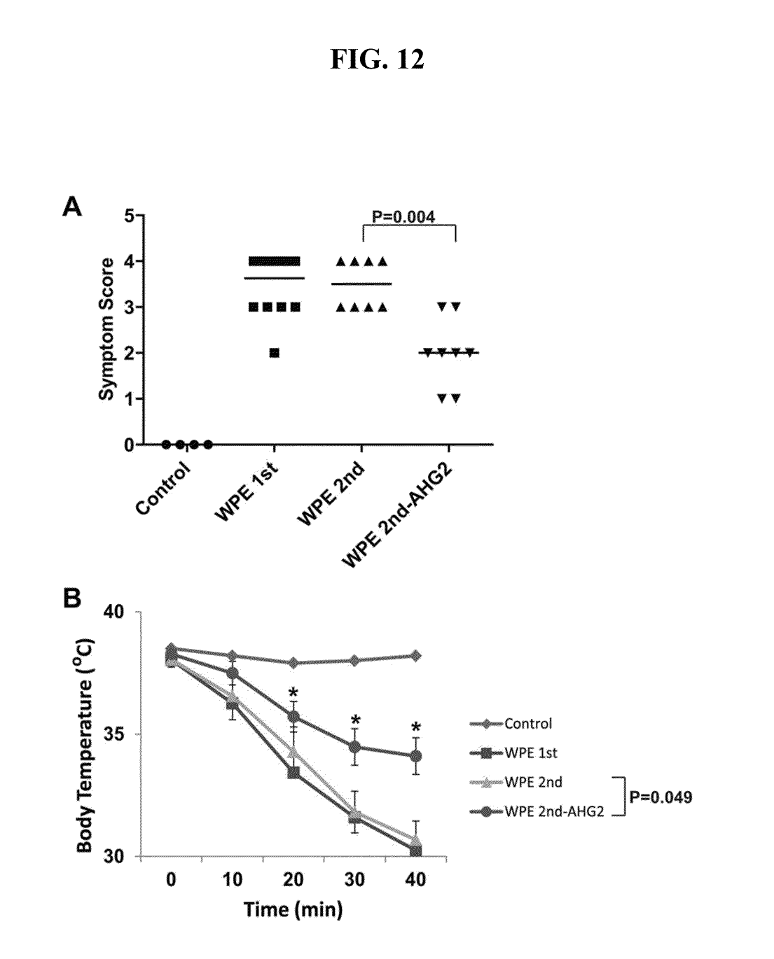

Core body temperatures of WPE challenged mice were also measured every 10 minutes. It was found that peanut allergen caused a severe acute anaphylactic reaction in WPE-sensitized mice (FIG. 5A) while AHG2 fusion protein itself did not cause any symptoms. When the sensitized mice were treated with AHG2 protein, their symptom scores were significantly improved (FIG. 5A). AHG2 also modestly inhibited the drop of body temperature in sensitized mice upon WPE challenge (FIG. 5B). The AHG2-treated mice also had lower levels of histamine than untreated mice (FIG. 5C). When peanut-challenged mice were treated with different doses of AHG2 and challenged again with WPE, AHG2 also showed inhibition of WPE-induced anaphylaxis (see FIG. 12). To determine whether the AHG2-induced inhibition is mediated by the inhibitory receptor FcγRII, AHG2 was tested in FcγRII deficient mice ¹⁹. Using the same procedures as before, the FcγRII KO mice were orally sensitized. When sensitized mice were challenged with WPE, it was found that the anaphylactic reaction exhibited by the mice was enhanced. AHG2 did not block either WPE-induced anaphylaxis (FIG. 6A) or the drop of body temperature in FcγRII deficient mice (FIG. 6B).

### AHG2 Blocked Peanut-Induced Inflammation in Airways

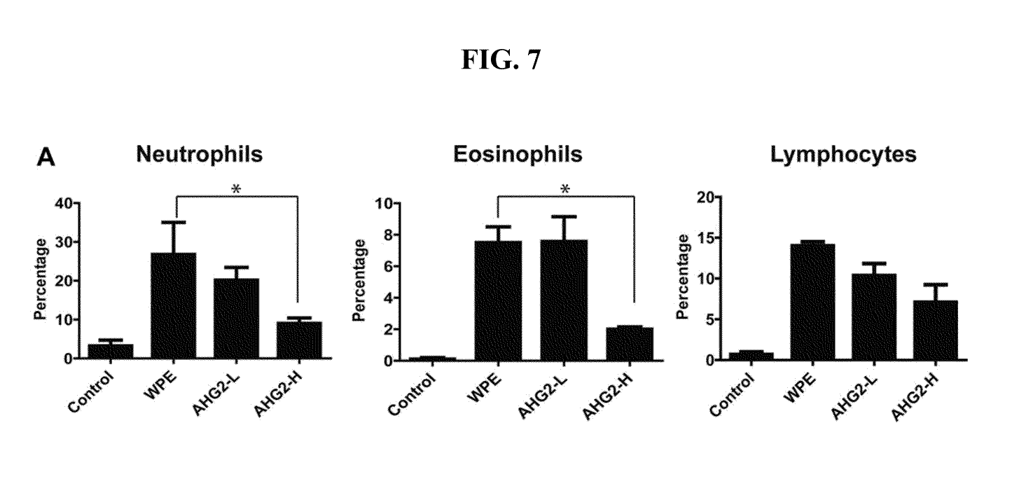

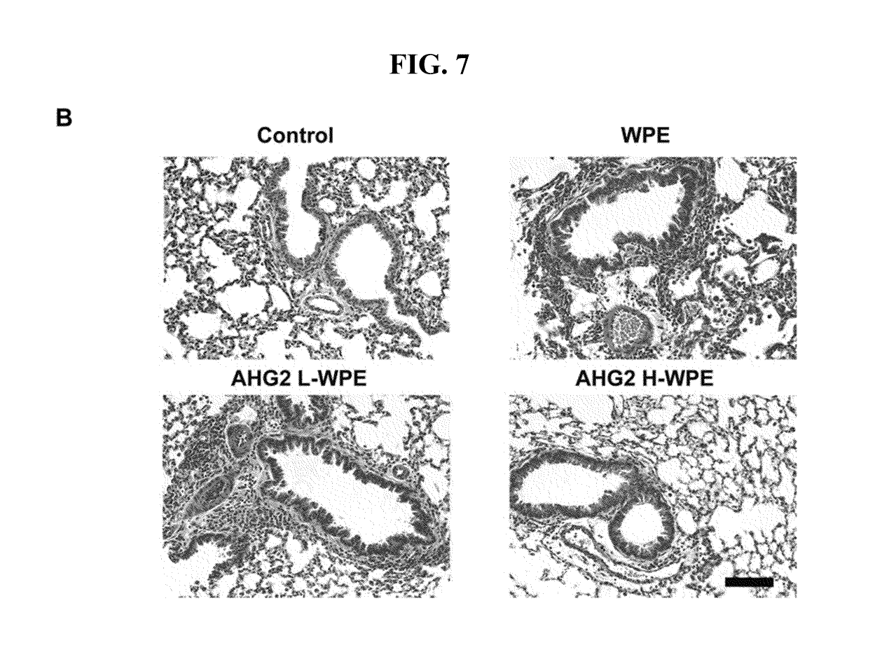

Peanut-induced acute anaphylaxis is often associated with airway inflammation ²⁰. To observe WPE-induced airway inflammation, bronchoalveolar lavages (BAL) and lung tissues was collected from WPE-sensitized mice after a WPE challenge. Histological evaluation showed that there were marked inflammatory infiltrates in the airways of WPE challenged mice. Differential cell counts in BAL showed that AHG2 fusion protein significantly inhibited antigen-induced neutrophil and eosinophil infiltration (FIG. 7A), although the lymphocyte count did not show a significant difference. The AHG2 protein decreased inflammatory infiltration induced by WPE in lung tissue as well (FIG. 7B).

### DISCUSSION

This Example describes the peanut allergen-Fcγ fusion protein's ability to block severe peanut-induced anaphylaxis. It was demonstrated that this fusion protein induced cross desensitization in the murine model of peanut allergy. This approach of using the major allergen in conjunction with Fcγ may be applied other allergens, besides peanut allergens.

In the systemic model, it was found that AHG2 did not completely block the WPE-induced anaphylactic reaction, unlike the previous tests in skin. While the present invention is not limited to any particular mechanism, and an understanding of the mechanism is not necessary to practice the present invention, it is possible that AHG2 is inactive in the systemic model due to the interference of mast cell independent or IgE independent mechanisms. Previous studies demonstrated that there are two pathways involved in systemic anaphylaxis in this model: the classic IgE-FIεRI-mast cell-mediated pathway and the alternate IgG-FcγRIII-macrophage-mediated pathway ²¹. When the amount of antigen is small, the IgE-dependent pathway is favored. However, larger amount of antigens readily induces the alternative pathway. Therefore, it may be that one could increase AHG2-induced inhibition by further decreasing the challenge allergen amount.

Unlike cat allergy, caused primarily by a single allergen Fel d1, there are as many as eleven proteins from peanuts that are identified as allergens. More than 90% of peanut hypersensitive individuals react to three main allergens—Ara h1, Ara h2, and Ara h3 ²². It is noted that each of these allerges, or allergic portions thereof, may be conjugated to Fcγ. In this Example, it was found that a fusion protein composed of the major peanut allergen Ara h2 and Fcγ effectively inhibited WPE-induced anaphylaxis and airway inflammation. In certain embodiments, in order to further suppress the allergic reaction, two or three or more of the following fusion proteins are used together—Ara h1-Fcγ, Ara h2-Fcγ, and Ara h3-Fcγ.

The results in this Example demonstrate that this fusion protein significantly inhibited WPE-induced peanut-specific IgE-mediated histamine release in human basophils as well as allergic responses in transgenic mice. Through the inhibitory receptor FcγRII, AHG2 also inhibited WPE-induced acute anaphylactic reaction and airway inflammation in the peanut allergy murine model. AHG2 itself did not induce anaphylaxis and inflammation in WPE-sensitized mice. While the present invention is not limited to any particular mechanism, this suggests that all properties of the allergen were mitigated by the presence of the FcγRIIb binding moiety.

### REFERENCES

1. Boyce J A, Assa'ad A, Burks A W, Jones S M, Sampson H A, Wood R A, et al. Guidelines for the Diagnosis and Management of Food Allergy in the United States: Summary of the NIAID-Sponsored Expert Panel Report. J Allergy Clin Immunol 2010;126:1105-18.
2. Leung D Y, Sampson H A, Yunginger J W, Burks A W, Jr., Schneider L C, Wortel C H, et al. Effect of anti-IgE therapy in patients with peanut allergy. N Engl J Med 2003;348:986-93.
3. Burks A W. Peanut allergy. Lancet 2008;371:1538-46.
4. Blumchen K, Ulbricht H, Staden U, Dobberstein K, Beschorner J, de Oliveira L C, et al. Oral peanut immunotherapy in children with peanut anaphylaxis. J Allergy Clin Immunol 2010;126:83-91.
5. Yu J W, Kagan R, Verreault N, Nicolas N, Joseph L, St Pierre Y, et al. Accidental ingestions in children with peanut allergy. J Allergy Clin Immunol 2006;118:466-72.
6. Maloney J M, Chapman M D, Sicherer S H. Peanut allergen exposure through saliva: assessment and interventions to reduce exposure. J Allergy Clin Immunol 2006;118:719-24.
7. Thyagarajan A, Varshney P, Jones S M, Sicherer S, Wood R, Vickery B P, et al. Peanut oral immunotherapy is not ready for clinical use. J Allergy Clin Immunol 2010;126:31-2.
8. Koppelman S J, Wensing M, Ertmann M, Knulst A C, Knol E F. Relevance of Ara h1, Ara h2 and Ara h3 in peanut-allergic patients, as determined by immunoglobulin E Western blotting, basophil-histamine release and intracutaneous testing: Ara h2 is the most important peanut allergen. Clin Exp Allergy 2004;34:583-90.
9. Finkelman F D. Peanut allergy and anaphylaxis. Curr Opin Immunol 2010;22:783-8.
10. Kalesnikoff J, Galli S J. New developments in mast cell biology. Nat Immunol 2008;9:1215-23.
11. Metcalfe D D, Peavy R D, Gilfillan A M. Mechanisms of mast cell signaling in anaphylaxis. J Allergy Clin Immunol 2009;124:639-46; quiz 47-8.
12. Daeron M, Malbec O, Latour S, Arock M, Fridman W H. Regulation of high-affinity IgE receptor-mediated mast cell activation by murine low-affinity IgG receptors. J Clin Invest 1995;95:577-85.
13. Jackman J, Chen Y, Huang A, Moffat B, Scheer JM, Leong SR, et al. Development of a two-part strategy to identify a therapeutic human bispecific antibody that inhibits IgE receptor signaling. J Biol Chem 2010;285:20850-9.
14. Zhu D, Kepley CL, Zhang M, Zhang K, Saxon A. A novel human immunoglobulin Fc gamma Fc epsilon bifunctional fusion protein inhibits Fc epsilon RI-mediated degranulation. Nat Med 2002;8:518-21.
15. Zhu D, Kepley C L, Zhang K, Terada T, Yamada T, Saxon A. A chimeric human-cat fusion protein blocks cat-induced allergy. Nat Med 2005;11:446-9.
16. Kepley C L, Cambier J C, Morel P A, Lujan D, Ortega E, Wilson B S, et al. Negative regulation of FcepsilonRI signaling by FcgammaRll costimulation in human blood basophils. J Allergy Clin Immunol 2000;106:337-48.

17. Dombrowicz D, Brini A T, Flamand V, Hicks E, Snouwaert J N, Kinet J P, et al. Anaphylaxis mediated through a humanized high affinity IgE receptor. J Immunol 1996;157:1645-51.
18. Sun J, Arias K, Alvarez D, Fattouh R, Walker T, Goncharova S, et al. Impact of CD40 ligand, B cells, and mast cells in peanut-induced anaphylactic responses. J Immunol 2007;179:6696-703.
19. Takai T, Ono M, Hikida M, Ohmori H, Ravetch J V. Augmented humoral and anaphylactic responses in Fc gamma Rh-deficient mice. Nature 1996;379:346-9.
20. Finkelman FD, Rothenberg M E, Brandt E B, Morris S C, Strait R T. Molecular mechanisms of anaphylaxis: lessons from studies with murine models. J Allergy Clin Immunol 2005;115:449-57; quiz 58.

21. Arias K, Chu D K, Flader K, Botelho F, Walker T, Arias N, et al. Distinct immune effector pathways contribute to the full expression of peanut-induced anaphylactic reactions in mice. J Allergy Clin Immunol 2011;127:1552-61 el.
22. McDermott R A, Porterfield H S, El Mezayen R, Burks A W, Pons L, Schlichting D G, et al. Contribution of Ara h 2 to peanut-specific, immunoglobulin E-mediated, cell activation. Clin Exp Allergy 2007;37:752-63.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for preventing or treating an allergic reaction to peanuts comprising:
administering a composition to a subject who has been exposed to a peanut allergen such that a peanut induced allergic reaction is reduced or eliminated in said subject, wherein said subject is or is suspected of being allergic to peanuts, and wherein said composition comprises a first fusion protein comprising: i) a first peanut allergen, or first allergenic portion thereof, and ii) an Fcγ1 protein, or functional portion thereof.

2. The method of claim 1, wherein said first peanut allergen comprises Ara h2 or an allergenic portion thereof.

3. The method of claim 1, wherein said peanut induced allergic reaction comprises peanut-induced anaphylaxis.

4. The method of claim 3, wherein administering reduces or eliminates said peanut-induced anaphylaxis in said subject.

5. The method of claim 1, wherein said peanut induced allergic reaction comprises airway inflammation.

6. The method of claim 5, wherein administering reduces or eliminates said airway inflammation in said subject.

7. The method of claim 1, wherein said composition further comprises a physiologically tolerable buffer.

8. The method of claim 1, wherein said composition is located in a self-administered injector.

9. A composition comprising: a first fusion protein comprising: i) a first peanut allergen, or first allergenic portion thereof, and ii) an Fcγ1 protein, or functional portion thereof

10. The composition of claim 9, wherein said first peanut allergen comprises Ara h2 or an allergenic portion thereof.

11. The composition of claim 9, wherein said composition further comprises a physiologically tolerable buffer.

12. A system comprising:
i) a composition comprising: a first fusion protein comprising: i) a first peanut allergen, or first allergenic portion thereof, and ii) an Fcγ1 protein, or functional portion thereof; and
ii) an injection device.

13. The system of claim 12, wherein said injection device comprises a self-administered injector.

14. The system of claim 12, wherein said first peanut allergen comprises Ara h2 or an allergenic portion thereof

15. The system of claim 12, wherein said composition further comprises a physiologically tolerable buffer.
